(19) United States
(12) Patent Application Publication
(10) Pub. No.: US 2026/0044681 A1
(43) Pub. Date: Feb. 12, 2026

(54) MULTI-HOP EVIDENCE PURSUIT FOR MEDICAL DECISION MAKING

(71) Applicant: NEC Laboratories America, Inc., Princeton, NJ (US)

(72) Inventor: Christopher Malon, Fort Lee, NJ (US)

(21) Appl. No.: 19/290,872

(22) Filed: Aug. 5, 2025

Related U.S. Application Data
(60) Provisional application No. 63/680,724, filed on Aug. 8, 2024.

(51) Int. Cl. G06F 40/40; G16H 10/60; G16H 70/20

(52) U.S. Cl. CPC G06F 40/40; G16H 10/60; G16H 70/20

(57) ABSTRACT

Methods and systems include generating a first question relating to supporting an input claim. A search is performed based on the first question to identify evidence relating to the input claim. An answer to the first question is generated based on the evidence. Additional questions are iteratively generated, with searches being performed based on the additional questions, and with answers to the additional questions being generated until a predetermined stop condition is reached. The input claim is classified by predicting a label based on evidence identified by the searches.

### RELATED APPLICATION INFORMATION

This application claims priority to U.S. Patent Application No. 63/680,724, filed on Aug. 8, 2024, incorporated herein by reference in its entirety.

### BACKGROUND

### Technical Field

The present invention relates to natural language queries searching and, more particularly, to evidence pursuit.

### Description of the Related Art

Natural language search accepts inputs from users in the form of a written query. Such searching may be enhanced by the use of large language models (LLMs) which can apply machine-based reasoning to the query and to the results to better identify and satisfy the user's intent.

### SUMMARY

A method includes generating a first question relating to supporting an input claim. A search is performed based on the first question to identify evidence relating to the input claim. An answer to the first question is generated based on the evidence. Additional questions are iteratively generated, with searches being performed based on the additional questions, and with answers to the additional questions being generated until a predetermined stop condition is reached. The input claim is classified by predicting a label based on evidence identified by the searches.

A system includes a hardware processor and a memory that stores a computer program. When executed by the hardware processor, the computer program causes the hardware processor to generate a first question relating to supporting an input claim, to perform a search based on the first question to identify evidence relating to the input claim, to generate an answer to the first question based on the evidence, to iteratively generate additional questions, perform searches based on the additional questions, and generate answers to the additional questions until a predetermined stop condition is reached, and to classify the input claim by predicting a label based on evidence identified by the searches.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block diagram illustrating an evidence pursuit system that iteratively generates questions about a claim to guide a search for evidence, in accordance with an embodiment of the present invention;

FIG. 2 is a block of pseudo-code that generates questions and answers to verify a claim, in accordance with an embodiment of the present invention;

FIG. 3 is a block of pseudo-code that searches for evidence based on a question relating to an input claim, in accordance with an embodiment of the present invention;

FIG. 4 is a block/flow diagram that collects evidence to support or refute a claim in furtherance of some downstream task, in accordance with an embodiment of the present invention;

FIG. 5 is a block diagram of a healthcare facility that includes an information system with evidence discovery that can support or refute an input claim based on medical information, in accordance with an embodiment of the present invention;

FIG. 6 is a block diagram of a computing device that can identify evidence to support or refute an input claim, in accordance with an embodiment of the present invention;

FIG. 7 is a diagram of an exemplary neural network architecture that can be used to implement part of a language model, in accordance with an embodiment of the present invention; and

FIG. 8 is a diagram of an exemplary neural network architecture that can be used to implement part of a language model, in accordance with an embodiment of the present invention.

### DETAILED DESCRIPTION OF PREFERRED EMBODIMENTS

A query to a natural language search system may include a factual statement and a request to verify that statement. Such a system may be used to fact-check information, to verify a person's assumptions, or to find support for a position. To that end, a sequence-to-sequence model can generate an initial question based on the input statement. A large language model (LLM) generates follow-up questions to pursue any evidence that is missing. A large language model (LLM) may be used to formulate answers to the questions one at a time, with a final answer being provided along with the supporting evidence that was discovered.

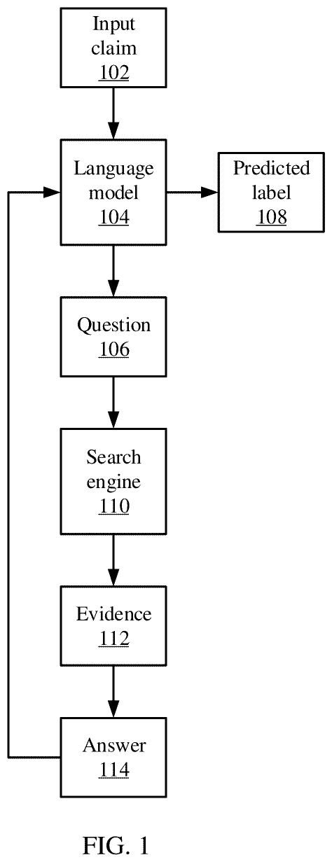

Referring now to FIG. 1, a block diagram of a natural language search is shown. An input claim 102 is processed by a language model 104 to generate a question 106 that seeks information about the claim. The question 106 is then used by a search engine 110 to identify one or more documents that provide evidence 112 for an answer 114 to the question. Although it is specifically contemplated that the search engine 110 may be a publicly accessible internet search engine, in some embodiments the search engine 110 may access an internal network or database instead of, or in addition to, searching the internet. The language model 104 may be used to generate the answer 114 to question 106 based on the evidence 112.

The answer 114 and the evidence 112 are further used by the LLM 104 to generate a next question. This process repeats until the language model 104 reaches a predetermined measure of confidence that the initial claim 102 is supported or not. The language model 104 generates predicted label 108 that identifies whether the claim 102 is supported and indicates the evidence 112 that has been collected to support the claim 102 or to disprove it. The generation of new questions 106 can stop when the language model 104 determines that it can classify the claim 102 as being supported or refuted without a follow-up question. The previously generated questions 106 are then paraphrased using an LLM and corresponding answers are found using the collected evidence 112 until a target number of question-answer pairs is reached.

In some embodiments, the language model 104 may be implemented as an LLM. In some embodiments, the language model 104 may be implemented as a sequence-to-sequence encoder-decoder transformer, such as T5. The system generates questions 106 before the search engine 110 operates to help steer the verification process. It is not assumed that all evidence can be found with a single search query, and so the current set of evidence is considered to generate any follow-up questions that may be appropriate to generate search queries for evidence that is missing.

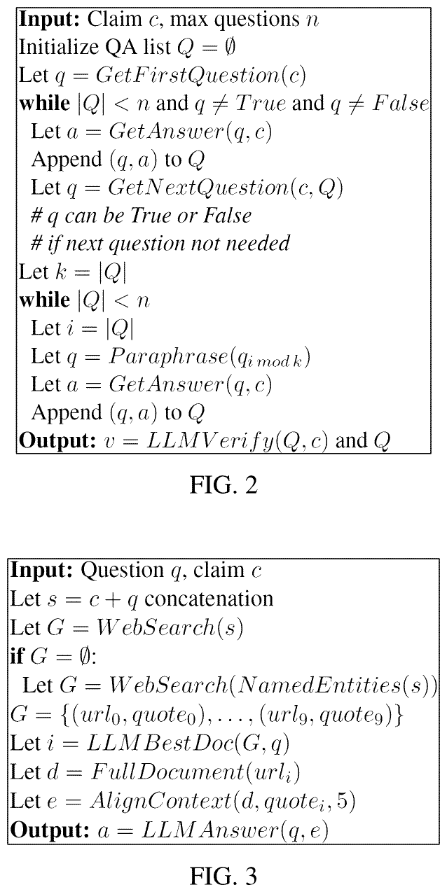

Referring now to FIG. 2, pseudo-code for verifying a claim is shown. The functions GetFirstQuestion and GetNextQuestion use the language model 104 to generate questions 106. In some embodiments these functions may be implemented by fine-tuning a sequence-to-sequence encoder-decoder transformer model. For GetFirstQuestion, the input is the claim 102 and the output is a first question 106. For GetNextQuestion, the input is the claim, concatenated with all previous question-answer pairs, and the output is the next question 106.

The prompt for GetNextQuestion may include a sequence of terms in the format, “Claim: <claim>,” “Question: <question0>,” “Answer: <answer0>,” “Question: <question1>,” “Answer: <answer1>.” The language model 104 may be fine-tuned using a set of predetermined question-answer pairs from a training dataset.

In some embodiments, the GetFirstQuestion and GetNextQuestion functions may be implemented using an LLM, prompted with similar inputs. Because the LLM output may be verbose and may include unnecessary explanations, the output may be split into sentences, of which the first sentence containing a question mark may be used as the output question. If this is not possible, then the entire output may be used instead.

In some cases, the next question may be a “true” or “false,” which indicates that the claim has been verified or refuted and processing can stop. Processing may also halt after a predetermined number of questions n have been generated.

The Paraphrase function asks the LLM for paraphrases of existing questions. In practice, multiple paraphrases of each question may be requested at once to avoid repeated calls, even though they may be used one at a time. Although the paraphrases may not be logically necessary once GetNextQuestion has determined a verdict, sometimes they provide a chance to reconsider the same questions using multiple sources. The LLMVerify function is called after all question-answer pairs are collected to choose the predicted label 108 for each example. Using additional question-answer pairs, LLMVerify may override the decision that stopped the question-generating process. For example, LLMVerify has a different prompt than GetNextQuestion and also considers the paraphrased question-answer pairs. The LLMVerify output therefore controls the system's answer, while GetNextQuestion is used to stop the question generating loop when it generates a “true” or “false” output.

The predicted label 108 may include various values, including “supported” to indicate that the claim is supported by the evidence, “refuted” to indicate that the claim is contradicted by the evidence, “not enough evidence” to indicate that there isn't enough evidence to form a conclusive response, and “conflicting evidence” or “cherrypicking” to indicate that there is substantial evidence both for and against the claim. In some embodiments, the early decision “true” or “false” from GetNextQuestion determines the final answer even after paraphrases, unless the loop has been performed the predetermined number of times without “true” or “false” being generated, in which case LLMVerify is called for the final answer. Label accuracy may be improved by considering the entire question and answer chain from the beginning when determining the label.

In some embodiments, GetFirstQuestion and GetNextQuestion may be implemented using different language models. For example, GetFirstQuestion may be implemented using the sequence-to-sequence model and GetNextQuestion may be implemented using the LLM. This may provide superior performance. Experiments show that the first question may be better as a rephrasing of the input claim, which the sequence-to-sequence can learn well, whereas a more general purpose LLM may attempt to generate unnecessarily complicated first questions. The generation of the subsequent questions, meanwhile, may call for deeper reasoning using the previously obtained answers, which may be better suited for an LLM.

Referring now to FIG. 3, pseudo-code is shown for generating an answer to a question. This pseudo-code may implement the GetAnswer function from FIG. 2, which retrieves evidence and uses it to answer the generated questions. The generated question is concatenated to the claim to form a web search query, and the top ten search results may be collected, including their URL (uniform resource locator), a short snippet displayed in the search results, and a page title, site name, and publication date. When a web search returns no results, the search may be repeated using only named entities from the initial search query and other capitalized words after the first word of the question.

By prompting, the function LLMBestDoc may be used to choose one document that best answers the question from the set of web search hits. The full text of the document may be retrieved and, if successful, a small window of text (e.g., five sentences) is selected that best overlaps the web search snippet using the AlignContext function. For example, all five-sentence windows of the document that include more than a threshold percentage (e.g., 70%) of the words in the web search snippet are recorded in order and the middle such window is taken. Using this window as the document excerpt provides more background and context to the text that the web search found to be relevant, while avoiding prompting with an overwhelming amount of text that might be found in a full web page. If the text extraction fails, the next stage may use only the web search snippet to represent the document text.

If a best document is selected and the text extraction succeeds, an LLM is prompted by LLMAnswer to answer the question using the selected window of text. If selecting a best document or text extraction fails, LLM answer can be executed using the text of the web search snippet only. If LLMBestDoc fails to select a best document, the full text of the LLM response may be used as the answer and the web search result page itself may be used as the evidence.

In the functions LLMBestDoc and LLMAnswer, the prompt may include not only the text for each document, but also metadata including the page title, site name, and publication data, when this metadata is included in the web search results. This metadata may be useful to assess the credibility or relevance of the information to the question.

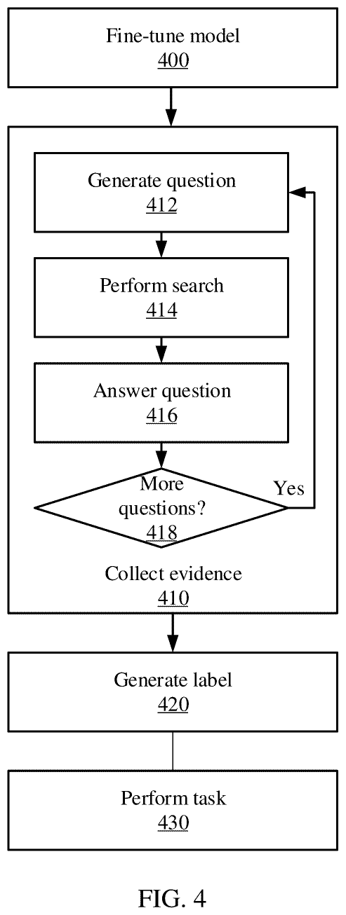

Referring now to FIG. 4, a method of finding evidence for or against a claim is shown. Block 400 begins by fine-tuning the language model 104 to perform the question generation task, using a dataset in which sequences of question-answer pairs are provided corresponding to claims. In the case of a sequence-to-sequence model, this finetuning may take the form of training with cross-entropy loss against the given questions.

Block 410 collects evidence for a given claim 102. As described above, block 412 generates a question 106 using the language model 104. Block 414 uses the question 106 to perform a search using search engine 110. Based on the results, block 416 answers the question using the results of the search as input to an LLM.

Block 418 then determines whether to generate more questions. As shown in the pseudo-code of FIG. 2, this may be based on whether a determination has been reached on whether the claim is supported or refuted, but may also be based on a number of questions already generated relative to a predetermined maximum. If more questions are called for, processing returns to block 412 to generate the next question.

Based on the list of generated questions, their answers, and the associated evidence, block 420 generates a label 108 for the claim 102, which may indicate whether the claim is supported or refuted by the evidence. Using this information, block 430 may then perform a task responsive to the label. In some cases, the task may involve interacting with the user in the form of a chatbot or other interactive information system. The task may include fact-checking claims in real-time or finding sources for a position. In a medical context, the task may include answering a user's medical questions and providing evidence-based advice.

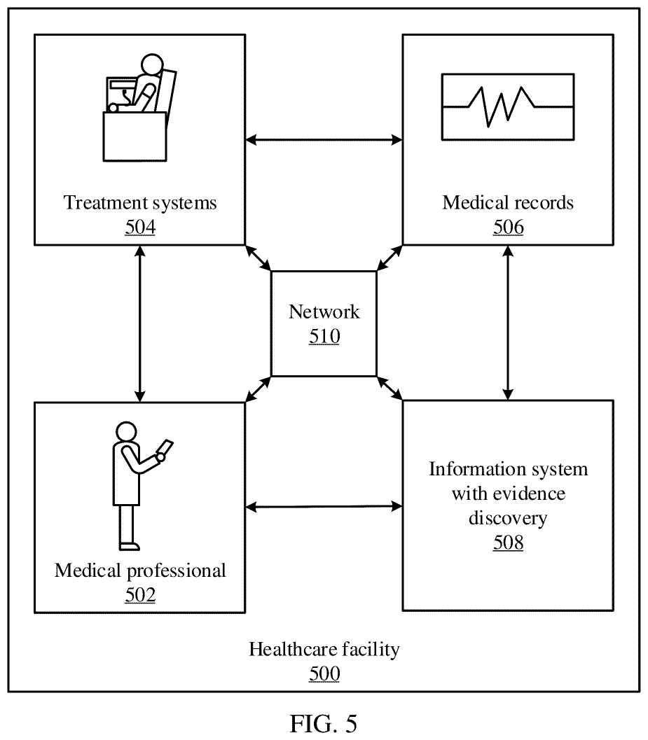

Referring now to FIG. 5, a diagram of time series analysis is shown in the context of a healthcare facility 500. An information system with evidence discovery 508 may be used to verify a claim and identify evidence to support the claim. In some cases the claim may relate to a patient's health condition, and a search engine of the information system 508 may refer to publicly available health information as well as the patient's medical records 506.

The healthcare facility may include one or more medical professionals 502 who review information extracted from a patient's medical records 506 to determine their healthcare and treatment needs. These medical records 506 may include self-reported information from the patient, test results, and notes by healthcare personnel made to the patient's file. Treatment systems 504 may furthermore monitor patient status to generate medical records 506 and may be designed to automatically administer and adjust treatments as needed.

Based on information drawn from the information system 508, the medical professionals 502 may then make medical decisions about patient healthcare suited to the patient's needs. For example, the medical professionals 502 may make a diagnosis of the patient's health condition and may prescribe particular medications, surgeries, and/or therapies that are appropriate to the stage of a disease.

The different elements of the healthcare facility 500 may communicate with one another via a network 510, for example using any appropriate wired or wireless communications protocol and medium. Thus the information system 508 receives data from treatment systems 504, medical professionals 502, and from medical records 506, and searches the medical records 506 to identify evidence that supports or contradicts an input claim. The information system 508 may further coordinate with treatment systems 504 in some cases to automatically administer or alter a treatment. For example, if the information system 508 finds evidence to support a claim that a given therapy is dangerous, or that the patient may have a particular health condition, the information system may trigger a change in treatment, such as initiating or halting the administration of a medication.

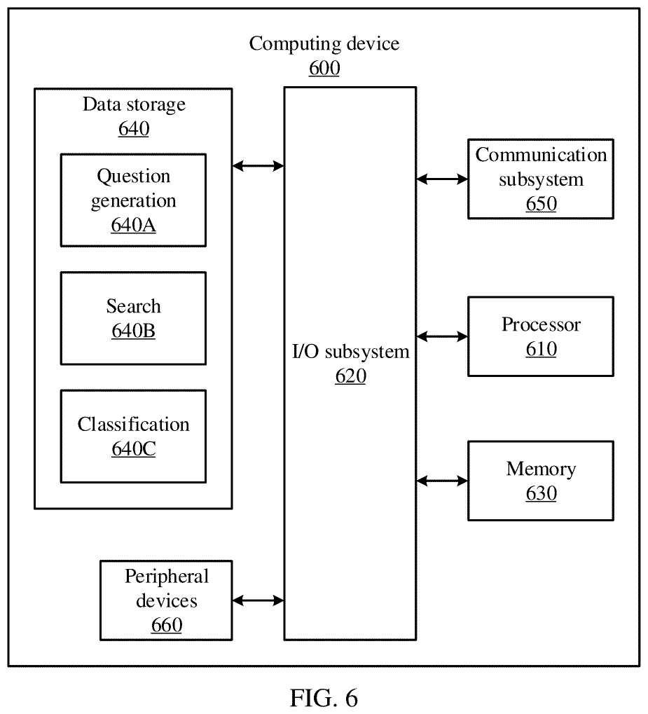

Referring now to FIG. 6, an exemplary computing device 600 is shown, in accordance with an embodiment of the present invention. The computing device 600 is configured to perform visual question answering.

The computing device 600 may be embodied as any type of computation or computer device capable of performing the functions described herein, including, without limitation, a computer, a server, a rack based server, a blade server, a workstation, a desktop computer, a laptop computer, a notebook computer, a tablet computer, a mobile computing device, a wearable computing device, a network appliance, a web appliance, a distributed computing system, a processor-based system, and/or a consumer electronic device. Additionally or alternatively, the computing device 600 may be embodied as one or more compute sleds, memory sleds, or other racks, sleds, computing chassis, or other components of a physically disaggregated computing device.

As shown in FIG. 6, the computing device 600 illustratively includes the processor 610, an input/output subsystem 620, a memory 630, a data storage device 640, and a communication subsystem 650, and/or other components and devices commonly found in a server or similar computing device. The computing device 600 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 630, or portions thereof, may be incorporated in the processor 610 in some embodiments.

The processor 610 may be embodied as any type of processor capable of performing the functions described herein. The processor 610 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 630 may be embodied as any type of volatile or non-volatile memory or data storage capable of performing the functions described herein. In operation, the memory 630 may store various data and software used during operation of the computing device 600, such as operating systems, applications, programs, libraries, and drivers. The memory 630 is communicatively coupled to the processor 610 via the I/O subsystem 620, which may be embodied as circuitry and/or components to facilitate input/output operations with the processor 610, the memory 630, and other components of the computing device 600. For example, the I/O subsystem 620 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 620 may form a portion of a system-on-a-chip (SOC) and be incorporated, along with the processor 610, the memory 630, and other components of the computing device 600, on a single integrated circuit chip.

The data storage device 640 may be embodied as any type of device or devices configured for short-term or long-term storage of data such as, for example, memory devices and circuits, memory cards, hard disk drives, solid state drives, or other data storage devices. The data storage device 640 can store program code 640A for generating questions and answers, 640B for searching for evidence, and/or 640C for classification of the input claim. Any or all of these program code blocks may be included in a given computing system. The communication subsystem 650 of the computing device 600 may be embodied as any network interface controller or other communication circuit, device, or collection thereof, capable of enabling communications between the computing device 600 and other remote devices over a network. The communication subsystem 650 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 600 may also include one or more peripheral devices 660. The peripheral devices 660 may include any number of additional input/output devices, interface devices, and/or other peripheral devices. For example, in some embodiments, the peripheral devices 660 may include a display, touch screen, graphics circuitry, keyboard, mouse, speaker system, microphone, network interface, and/or other input/output devices, interface devices, and/or peripheral devices.

Of course, the computing device 600 may also include other elements (not shown), as readily contemplated by one of skill in the art, as well as omit certain elements. For example, various other sensors, input devices, and/or output devices can be included in computing device 600, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 600 are readily contemplated by one of ordinary skill in the art given the teachings of the present invention provided herein.

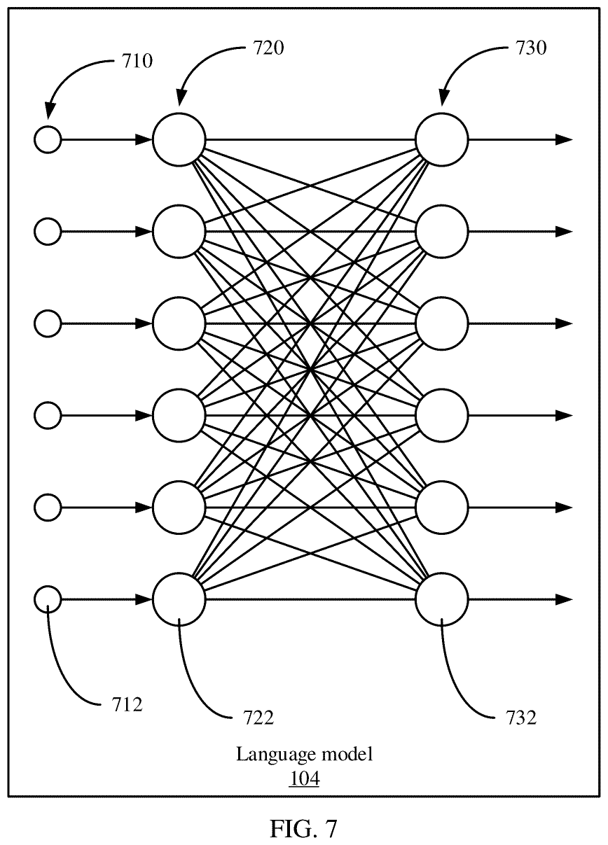

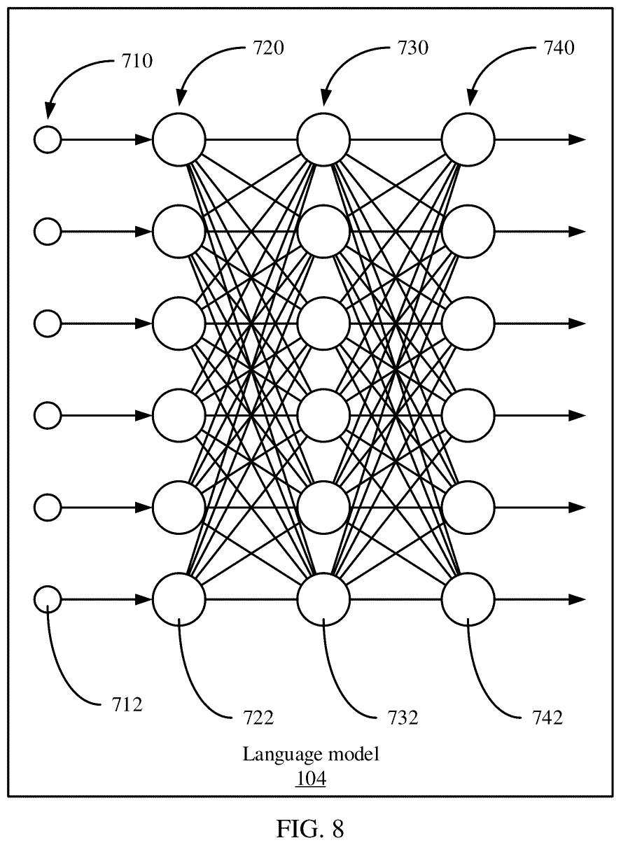

Referring now to FIGS. 7 and 8, exemplary neural network architectures are shown, which may be used to implement parts of the present machine learning models, such as the language model 104. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the input data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 720 of source nodes 722, and a single computation layer 730 having one or more computation nodes 732 that also act as output nodes, where there is a single computation node 732 for each possible category into which the input example could be classified. An input layer 720 can have a number of source nodes 722 equal to the number of data values 712 in the input data 710. The data values 712 in the input data 710 can be represented as a column vector. Each computation node 732 in the computation layer 730 generates a linear combination of weighted values from the input data 710 fed into input nodes 720, and applies a non-linear activation function that is differentiable to the sum. The exemplary simple neural network can perform classification on linearly separable examples (e.g., patterns).

A deep neural network, such as a multilayer perceptron, can have an input layer 720 of source nodes 722, one or more computation layer(s) 730 having one or more computation nodes 732, and an output layer 740, where there is a single output node 742 for each possible category into which the input example could be classified. An input layer 720 can have a number of source nodes 722 equal to the number of data values 712 in the input data 710. The computation nodes 732 in the computation layer(s) 730 can also be referred to as hidden layers, because they are between the source nodes 722 and output node(s) 742 and are not directly observed. Each node 732, 742 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . . wₙ₋₁, wₙ. The output layer provides the overall response of the network to the input data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 732 in the one or more computation (hidden) layer(s) 730 perform a nonlinear transformation on the input data 712 that generates a feature space. The classes or categories may be more easily separated in the feature space than in the original data space.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method, comprising:
generating a first question relating to supporting an input claim;
performing a search based on the first question to identify evidence relating to the input claim;
generating an answer to the first question based on the evidence;
iteratively generating additional questions, performing searches based on the additional questions, and generating answers to the additional questions until a predetermined stop condition is reached; and
classifying the input claim by predicting a label based on evidence identified by the searches.

2. The method of claim 1, wherein generating the first question is performed using a machine learning model that includes a sequence-to-sequence encoder-decoder transformer model.

3. The method of claim 2, wherein generating the additional questions is performed using a large language model.

4. The method of claim 1, wherein generating the additional questions can include an output of “true” or “false” and wherein the stop condition includes determining that a most recent question of the additional question is “true” or “false.”

5. The method of claim 1, further comprising extracting text from a document retrieved by the search as the evidence.

6. The method of claim 5, wherein extracting text from the document includes selecting a window of text that includes more than a threshold percentage of words from a search snippet.

7. The method of claim 1, further comprising paraphrasing the evidence identified by the searches, wherein classifying the input claim includes using a large language model to generate the label using an input that includes the first question, the additional questions, the answers, and the paraphrased evidence.

8. The method of claim 1, wherein generating the answers includes processing the question using a large language model, with the evidence as an input.

9. The method of claim 1, wherein the search is performed over public medical information and over a patient's medical records for use in medical decision making.

10. The method of claim 9, wherein the input claim is a claim relating to a medical condition of the patient, further comprising performing a treatment action responsive to the label.

11. A system, comprising:
a hardware processor; and
a memory that stores a computer program which, when executed by the hardware processor, causes the hardware processor to:
generate a first question relating to supporting an input claim;
perform a search based on the first question to identify evidence relating to the input claim;
generate an answer to the first question based on the evidence;
iteratively generate additional questions, perform searches based on the additional questions, and generate answers to the additional questions until a predetermined stop condition is reached; and
classify the input claim by predicting a label based on evidence identified by the searches.

12. The system of claim 11, wherein generation of the first question is performed using a machine learning model that includes a sequence-to-sequence encoder-decoder transformer model.

13. The system of claim 12, wherein generation of the additional questions is performed using a large language model.

14. The system of claim 11, wherein generation of the additional questions can include an output of “true” or “false” and wherein the stop condition includes a determination that a most recent question of the additional question is “true” or “false.”

15. The system of claim 11, wherein the computer program further causes the hardware processor to extract text from a document retrieved by the search as the evidence.

16. The system of claim 15, wherein extraction of text from the document includes selection of a window of text that includes more than a threshold percentage of words from a search snippet.

17. The system of claim 11, wherein the computer program further causes the hardware processor to paraphrase the evidence identified by the searches, wherein classification of the input claim includes using a large language model to generate the label using an input that includes the first question, the additional questions, the answers, and the paraphrased evidence.

18. The system of claim 11, wherein generation of the answers includes processing the question using a large language model, with the evidence as an input.

19. The system of claim 11, wherein the search is performed over public medical information and over a patient's medical records for use in medical decision making.

20. The system of claim 19. wherein the input claim is a claim relating to a medical condition of the patient. wherein the computer program further causes the hardware processor to perform a treatment action responsive to the label.
